(19) United States
(12) Patent Application Publication
(10) Pub. No.: US 2002/0119101 A1
(43) Pub. Date: Aug. 29, 2002

(54) MRI image enhancement compositions

(75) Inventors: Christopher Mark Perkins, Cincinnati, OH (US); David Johnathan Kitko, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 09/957,392

(22) Filed: Sep. 20, 2001

Related U.S. Application Data
(60) Provisional application No. 60/235,011, filed on Sep. 25, 2000.

(51) Int. Cl. A61B 5/055

(52) U.S. Cl. 424/9.36

(57) ABSTRACT

The present invention relates to pharmaceutical compositions and methods for using said compositions which comprise:

a) an effective amount of a magnetic resonance imaging agent, for example, a complex having the formula:

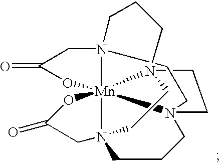

and

b) the balance carriers and other adjunct ingredients.

This Application claims priority to U.S. Provisional Pat. App. Ser. No. 60/235,011 filed Sep. 25, 2000.

### FIELD OF THE INVENTION

The present invention relates to magnetic resonance imaging (MRI) and to compositions comprising substituted 1,4,8,11-tetraazabicyclo[6.6.2] hexadecane manganese (II) complexes which are suitable for use as magnetic resonance imaging tools for medical diagnosis. The present invention also relates to methods for providing a magnetic resonance image which is suitable for use in medical diagnosis.

### BACKGROUND OF THE INVENTION

The availability of magnet resonance imaging (MRI) devices has led to the use of MRI in medical examinations for the detection and diagnosis of disease states and other internal abnormalities. The continued use and development of MRI has stimulated interest in the development of pharmaceutical agents capable of altering MRI images in diagnostically useful ways. Pharmaceutical agents (MRI pharmaceuticals) which are currently favored by researchers in the field are suitably complexed paramagnetic metal cations. The use of pharmaceuticals in MRI imaging offers major opportunities for improving the value of the diagnostic information which can be obtained.

Radiopharmaceuticals, which are used in radioisotopic imaging in a manner analogous to MRI pharmaceuticals, are a well-developed field. The knowledge existing in this field thus provides a starting point for the development of MRI pharmaceuticals. MRI pharmaceuticals must meet certain characteristics, however, which are either not required or are considerably less critical in the case of radiopharmaceuticals. MRI pharmaceuticals must be used in greater quantities than radiopharmaceuticals. As a result, they must not only produce detectable changes in proton relaxation rates but they must also be (a) substantially less toxic, thereby permitting the use of greater amounts, (b) more water soluble to permit the administration of a higher dosage in physiologically acceptable volumes of solution, and (c) more stable in vivo than their radiopharmaceutical counterparts. In vivo stability is important in preventing the release of free paramagnetic metals and free ligand in the body of the patient, and is likewise more critical due to the higher quantities used. For the same reasons, MRI pharmaceuticals which exhibit whole body clearance within relatively short time periods are particularly desirable.

Since radiopharmaceuticals are administered in very small dosages, there has been little need to minimize the toxicity of these agents while maximizing water solubility, in vivo stability and whole body clearance. It is not surprising therefore that few of the ligands developed for use as components in radiopharmaceutical preparations are suitable for use in preparation of MRI pharmaceuticals. A notable exception is the well known ligand diethylene triamine pentaacetic acid (DTPA), which has proved useful in forming complexes with both radiocations, pharmacologically suitable salts of which provide useful radiopharmaceuticals, and paramagnetic cations such as gadolinium, whose pharmacologically suitable salts have proved useful as MRI pharmaceuticals.

The contrast agents used in MRI derive their signal-enhancing effect from the inclusion of a material exhibiting paramagnetic, ferromagnetic, ferromagnetic, or superparamagnetic behavior. These materials affect the characteristic relaxation timers of the imaging nuclei in the body regions into which they distribute causing an increase or decrease in magnetic resonance signal intensity. There is therefore a long felt need for an MRI imaging agent which is substantially non-toxic, highly water soluble, and highly stable in vivo and which is capable of selectively enhancing signal intensity in particular tissue types.

### SUMMARY OF THE INVENTION

The present invention meets the aforementioned needs in that it has been surprisingly discovered that certain bicyclo manganese complexes, for example, substituted 1,4,8,11 -tetraazabicyclo[6.6.2] hexadecane manganese (II) transition metal complexes are preferential MRI imaging agents and are suitable for use in magnetic resonance imaging compositions which provide enhanced medical diagnostic information. It has been surprisingly discovered that by increasing the lipophilicity of MRI imaging agents, the ability to target different types of body tissue is greatly enhanced. The complexes of the present invention provide enhanced imaging of arteries and veins, as well as nephric tissue imaging.

The first aspect of the present invention relates to a pharmaceutical composition comprising:

a) from about 0.01% to about 99.99%, in another embodiment from 1 % to about 50%, wherein another embodiment comprises from about 10% to about 75% by weight, of a 1,4,8,11-tetraaza-bicyclo[6.6.2] hexadecane manganese (II) complex magnetic resonance imaging agent selected from the group:

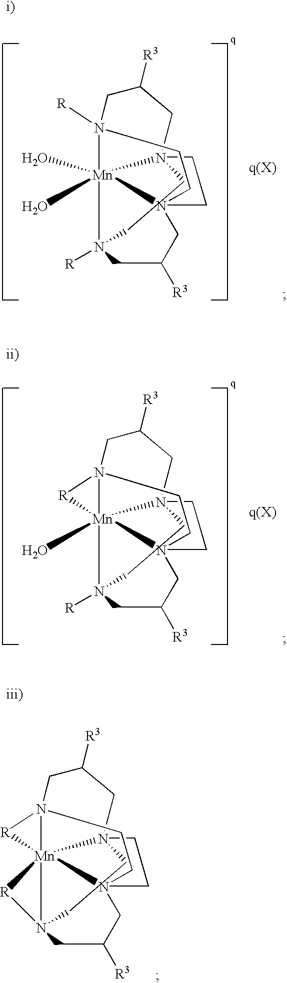

iv) and mixtures thereof;

wherein each R is independently selected from the group consisting of:

i) C₁—C₁₈ hydrocarbyl;

ii) —(CH₂)ₙCO₂—;

iii) CH₃(CH₂)ₙCO—;

iv) —(CH₂)ₙR¹;

v) —(CH₂)ₙOPO₃^{—};

vi) —[(CH₂)ₙOPO₃R²(phenyl)₂]^{—};

R¹ is hydroxyl, 2-hydroxyphenyl, 2-pyridyl, 2-furfuryl, and mixtures thereof; R² is C₁—C₁₂ linear, branched, or cyclic alkylene;

R³ is selected from the group consisting of:

i) hydrogen;

ii) C₁—C₁₈ hydrocarbyl;

iii) —OH;

iv) —(CH₂)ₘCO_{2—};

v) —O(CH₂)ₘCO_{2 —};

vi) and mixtures thereof;

the indices m and n have the value from 0 to about 10; X is an pharmaceutically compatible anion in sufficient amount q to provide electronic neutrality; and

b) the balance carriers and other adjunct ingredients.

The present invention further relates to methods for providing an enhanced magnetic resonance image contrast in human tissue, said method comprising the step of administering to a human an effective amount of a 1,4,8,11-tetraazabicyclo[6.6.2] hexadecane manganese (II) complex, preferably in a pharmaceutical composition further comprising one or more carriers and adjunct ingredients.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference.

### DETAILED DESCRIPTION OF THE INVENTION

The present invention relates to 1,4,8,11 -tetraazabicyclo[6.6.2] hexadecane manganese (II) complexes, for example, 4,11-dimethyl-1,4,8,11-tetraazabicyclo[6.6.2] hexadecane manganese (II) diaquo complex which is a magnetic resonance imaging (MRI) tissue-contrasting agent suitable for in vivo use by humans. The manganese metal complex of the present invention comprises a 1,4,8,11-tetraazabicyclo[6.6.2] hexadecane ligand which chelates the paramagnetic transition metal cation manganese (II) at four sites of the metal's coordination sphere. In one embodiment, the remaining 2 sites are can be solvated by water or comprise one or more other non-reactive ligands, in another embodiment, one remaining site is occupied by a unit which is further bonded to a bicyclo ring nitrogen, and in a third variation, both available sites are occupied by a unit which is further bonded to a bicyclo ring nitrogen.

The term “hydrocarbyl” relates to any hydrocarbon chain having from 1 to 18 carbon atoms. The chains may be linear, inter alia, octyl, and decyl; or branched, inter alia, 6-methyl octyl. The chains may be acyclic; alkyl, alkenyl, alkynyl, and the like, or cyclic, for example, cyclohexyl, or bicyclo [2.2.1]heptanyl. The term hydrocarbyl also encompasses any type of chain branching of the units such that the total number of carbon atoms in said chain is from 1 to 18. Hydrocarbyl units may be aromatic or non-aromatic.

The first aspect of the present invention relates to MRI contrasting agents in a composition comprising a suitable carrier or other adjunct ingredient.

The first embodiment of this aspect relates to compounds having the formula:

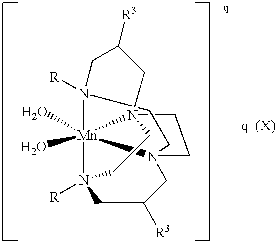

wherein there are two free sites which are capable of forming the diaquo species in situ.

The second embodiment of this aspect relates to compounds having the formula:

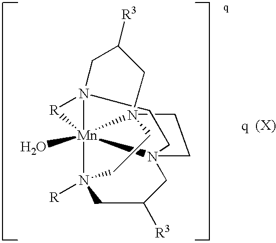

wherein one of the free sites is occupied by a ligand which is attached to a bicyclo ring nitrogen.

The third embodiment of this aspect relates to compounds having the formula:

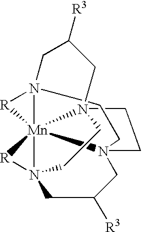

wherein both of the free sites are occupied by a ligand which is attached to a bicyclo ring nitrogen.

Each R unit is independently selected from the group consisting of:

i) C₁—C₁₈hydrocarbyl;

ii) —(CH₂)ₙCO_{2—;}

iii) CH₃(CH₂)ₙCO—;

iv) —(CH₂)ₙR¹;

v) —(CH₂)ₙOPO₃^{—};

vi) —[(CH₂)ₙOPO₃R²(phenyl)₂]^{—};

wherein R¹is hydroxyl, 2-hydroxyphenyl, 2-pyridyl, 2-furfuryl; and mixtures thereof; R² is C₁—C₁₂ linear, branched, or cyclic alkylene.;

R³ is a bicyclo ring substituent and is selected from the group consisting of:

i) hydrogen;

ii) C₁—C₁₈ hydrocarbyl;

iii) —OH;

iv) —(CH₂)ₘCO₂—;

v) —O(CH₂)ₘCO₂—;

vi) and mixtures thereof.

The indices m and n each independently have the value from 0 to about 10; X is a pharmaceutically compatible anion in sufficient amount q to provide electronic neutrality. In one embodiment n from 1 to 4. The present invention also relates to an embodiment wherein the index n is 1 or 2. The index m is equal to 1 in one embodiment wherein R³ comprises a —CH₂CO₂^{—}unit. X is any suitable anion in an amount q which is sufficient amount to satisfy electronic neutrality. Non-limiting examples of X include chlorine, bromine, nitrate, sulfate, carbonate, phosphate, hexafluorophosphate, tetrafluoroborate, and mixtures thereof.

One embodiment of the present invention relates to R units which are methyl, ethyl, isopropyl, butyl, and mixtures thereof.

It has now been surprisingly found that when used to contrast hepatic tissue compounds having the formula:

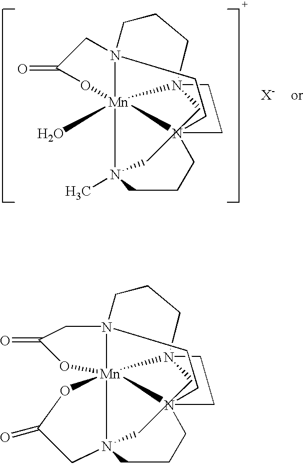

are better than Gd(DTPA) in relaxing irradiated proton spins.

Another aspect of the present invention relates to contrasting agents wherein one or both R units are α,α-biphenyl phosphate units having the formula:

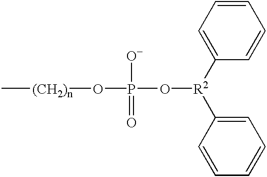

wherein R¹ is a C₁—C₁₂ linear, branched, or cyclic alkylene spacer unit having two phenyl groups attached thereto. In one embodiment, R has the formula:

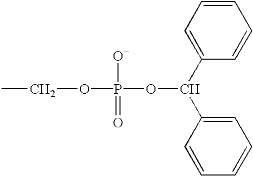

while in another embodiment R has the formula:

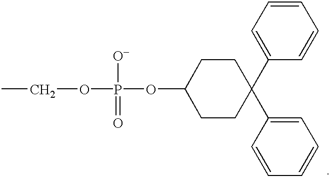

The following are non-limiting examples of suitable ligand for use in the contrasting agents of the present invention.

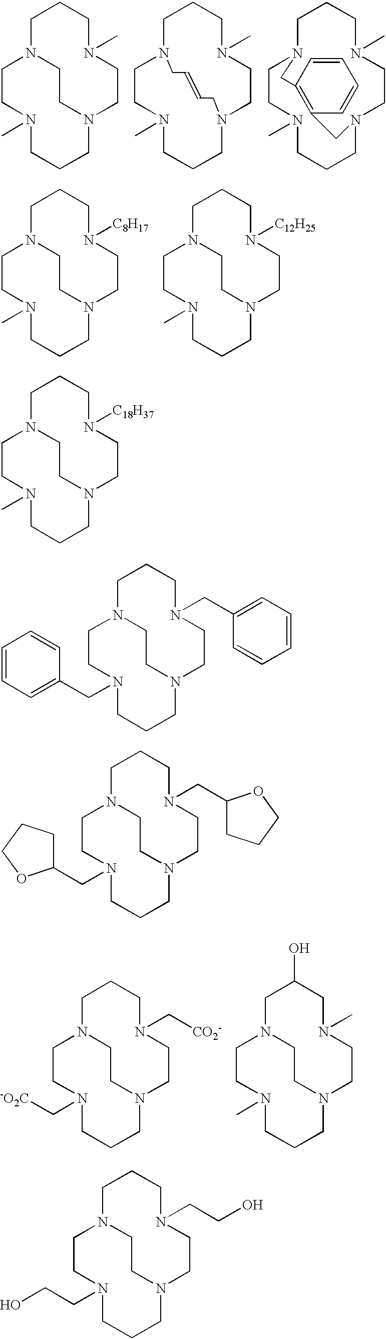

The following is a non-limiting example of a procedure for preparing the 4,11 -dimethyl-1,4,8,11 -tetraazabicyclo[6.6.2] hexadecane ligand.

Preparation of 4 ,11 -dimethyl-1 ,4 ,8,11 -tetraaza-bicyclo[6.6.21] hexadecane

To a 250 mL, 3 necked round bottom flask, equipped with a thermometer, nitrogen inlet, and magnetic stirrer is added N,N′-bis(2-aminoethyl)-1,3-propanediamine (5.00 g, 31.3 mmol) and absolute ethanol (100 mL). The solution is stirred under argon and cooled to 15° C. using an ice bath. Aqueous glyoxal (4.78 g., 33 mmol, 40% in water) is added dropwise with stirring. Upon completion of the addition, the solution is concentrated under reduced pressure to yield a clear, colorless oil. The isolated oil has the formula:

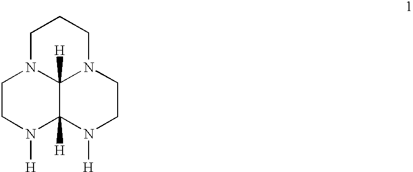

and is obtained in 100% yield (6.0 g).

Cyclic amine 1 (6.0 g) is suspended in acetonitrile (100 mL). Potassium carbonate (25 g) and 1,3-propanediol ditosylate (12.61 g, 32.8 mmol) are added. The solution is stirred vigorously at RT overnight. The reaction is then warmed to 70° C. and filtered hot with glass fiber filter paper and vacuum filtration. The resulting solid is washed with acetonitrile (100 mL). The acetonitrile filtrate is concentrated under reduced pressure to yield a light green oil having the formula:

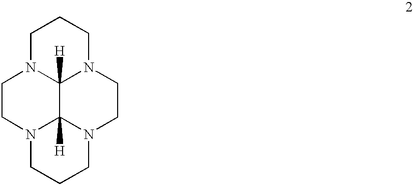

and is obtained in 100% yield (7.0 g).

The tetraamine 2, (7.0 g) is dissolved in acetonitrile (150 mL). Methyl sulfate (2.5 equiv.) is added, the reaction warmed to 65° C. and stirred for 9 days. The solvent is removed under reduced pressure to yield a brown oil having the formula:

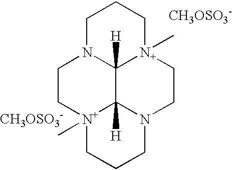

and is obtained in approximately 85% yield.

Distilled water (25 mL) and potassium carbonate (13.8 g) are added to a 250 mL round bottomed flask. Absolute ethanol (75 mL) is added and the resulting biphasic solution is stirred and heated to 60° C. with an oil bath. Sodium borohydride (1.60 g., 42.3 mmol) and 3 (10.0 g., 21.1 mmol) was added to the solution. The reaction is stirred at 60° C. for 75 minutes. The reaction mixture is placed in a separatory funnel and the ethanol layer collected. The solvent is then removed under reduced pressure, the resulting tan solid/oil is dissolved in 5N KOH (5 mL) and extracted with toluene (2 ×50 mL). The toluene is removed under reduced pressure to yield 4,11-dimethyl-1,4,8,11-tetraaza-bicyclo[6.6.2]hexadecane having the formula:

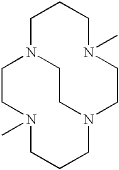

as an oil, in 95% yield (5.2 g) after distillation.

4-carboxymethyl-11-methyl-1,4 ,8, 11 -tetraaza-bicyclo[6.6.2]hexadecane.

Tetraamine 2 (64.6 g.) is dissolved in dry acetonitrile (500 mL). Benzyl bromide (42.75 g.) is added to the stirred solution under Ar. The solution is stirred at room temperature for 3 days. Methyl iodide (248 g., 1.75 mol) is added and the solution is stirred an additional 3 days. The reaction is then filtered using Whatman #4 paper and vacuum filtration. The solid 1s washed with CH₃CN (200 mL). A white solid, having the formula

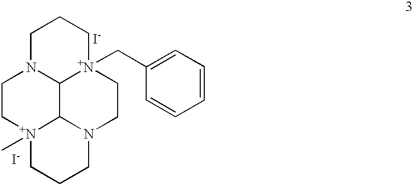

resulted in 86% yield (115.8 g.).

Diquat, 3,115 g and 35 g of potassium carbonate are dissolved in 1 L 75% ethanol in water. This solution is warmed to 50° C. and sodium borohydride (16.5 g) is added over 15 minutes. After stirring 1 hr, the reaction is pH-adjusted to 6.0 with concentrated hydrochloric acid and then evaporated to dryness. The residue is dissolved in 5 N potassium hydroxide and extracted 6 times with 200 mL portions of toluene. The toluene extracts are combined and dried with sodium sulfate, filtered and evaporated to dryness then dried under 0.05 mm vacuum overnight. This results in an oil having the formula

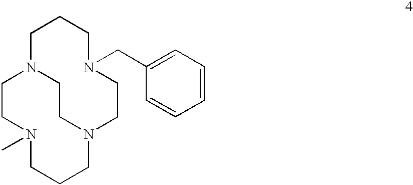

in 92% yield (65.3 g.).

Benzyl-methyl bridged cyclam, 4, (15 g.) is dissolved in ethanol (150 mL) and hydrogenated using 1.5 g. 20% palladium hydroxide on carbon at 50 psi for 18 hours. The reaction is filtered and evaporated to dryness to an oil having the formula

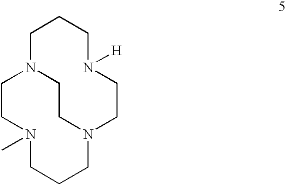

in 100% yield (11 g.).

Hydro methyl bridged cyclam, 5, (5.00 g.) and potassium carbonate (20 g.) are dissolved in acetonitrile (125 mL). Ethyl 2-iodoacetate (4.45 g) is added dropwise, under argon, with stirring over 5 minutes. After 2 hours stirring at room temperature, the solution is filtered and evaporated to dryness yielding a yellow-orange solid having the formula

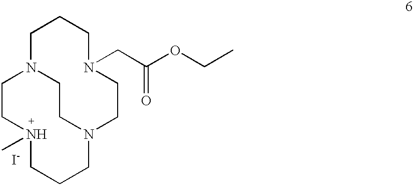

in 100% yield (9.3 g.).

The hydroiodide salt, 6, (9.3 g.) is stirred with 50 mL of Amberlite IRA-400 resin (OH—) in 100 mL of water overnight. The resin was filtered off and the filtrate evaporated to dryness resulting in a white solid having the formula

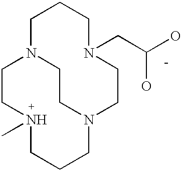

100% yield (7.05 g as the dihydrate).

The carboxymethyl methyl bridged cyclam (2.5 g.) was slurried in acetonitrile (50 mL) and the solution was degassed by applying a vacuum to the room temperature solution until it boiled and then venting with argon (repeated six times). The solution was warmed to reflux forming a clear, colorless solution. Anhydrous manganese chloride (1.00 g.) was added and the reaction was refluxed 1 hour. Solids began forming on the flask so an additional 40 mL of degassed anhydrous acetonitrile was added. The reaction was stirred at reflux overnight, then cooled to room temperature forming solids. These solids were re-dissolved in hot 3:1 acetonitrile/methanol and filtered using a 0.2 μ membrane filter. The clear solution was evaporated to dryness producing a light yellow solid of the formula

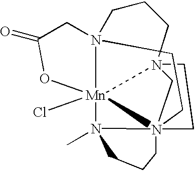

80% yield (2.57 g.).

Preparation of 4 ,11 -Bis(carboxymethyl)-1 ,4,8,11 -Tetraaza-bicyclo[6.6.2] hexadecane Manganese(II) 1 ,4,8,11 -tetraaza-bicyclo-[6.6.2] hexadecane (1.5 g.) and potassium carbonate (7.5 g.) were dissolved in dry acetonitrile (125 mL). Ethyl 2-iodoacetate (3.12 g.) was added dropwise, under argon, with stirring over 10 minutes. After 3.5 hours stirring at room temperature, the solution was filtered and evaporated to dryness yielding a yellow-orange solid having the formula

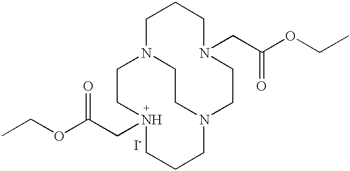

in 100% yield (3.2 g.).

The hydroiodide salt (3.2 g.) was stirred with 50 mL of Amberlite IRA-400 resin (OH—) in 100 mL of water overnight. The resin was filtered off and the filtrate evaporated to dryness resulting in a white solid having the formula

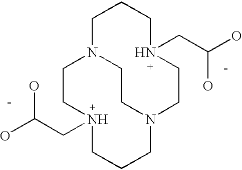

in 88% yield (2.1 g as the hydrate).

The bis(carboxymethyl) bridged cyclam (2.1 g.) was slurried in 4:1 acetonitrile/methanol (50 mL) and the solution was degassed by applying a vacuum to the room temperature solution until it boiled and then venting with argon (repeated six times). The solution was warmed to reflux forming a clear, colorless solution. Anhydrous manganese chloride (0.660 g.) was added to the reaction and a white precipitate immediately formed. The reaction was then refluxed 1.5 hours and then cooled to room temperature. The solids were filtered from the solution using a 0.2 μ membrane filter and dried overnight under 0.05 mm vacuum. This resulted in white solids having the formula

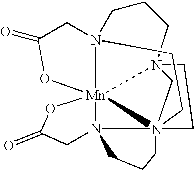

in 80% yield (1.3 g.).

Preparation of Dichloro 4 ,11 -Diethyl-1,4,8,11 tetraaza-bicyclo [6.6.2] hexadecane Manganese(II)

Tetraamine 2 (5.55 g.) was dissolved in dry acetonitrile (50 mL) and added with ethylbromide (34.5 g.) to a pressure flask. The reaction vessel was placed in an oil bath at 80° C. and stirred vigorously for 24 hr. The oil bath temperature was then elevated to 130° C. and the reaction was stirred for 6 additional hours. After cooling the reaction to RT, the brown solid having the formula

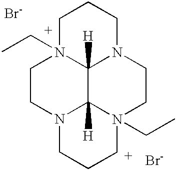

was filtered off and dried 69% yield (7.6 g.).

This material (3.0 g.) was then dissolved in 1 M potassium carbonate (30 mL) and added to a glass rocking autoclave sleeve along with 20% palladium hydroxide /carbon (0.7 g.). The glass sleeve was placed inside a rocking autoclave and hydrogenated at elevated temperature and pressure (65° C., 1900 psi hydrogen) for 4 hr, while rocking. The reaction was then cooled, vented, and the glass sleeve was removed from the autoclave. The reaction was then filtered through glass fiber filter paper to remove the catalyst and the filtrate was evaporated to dryness. Once dry, the white solids were suspended in refluxing ethanol (100 mL) for several minutes and filtered. The filtrate was evaporated to dryness and the oily residue dissolved in aqueous KOH (4 mL, 4 M) and extracted with toluene (3×25 mL). The toluene extracts were combined and evaporated to dryness to yield a clear oil having the formula

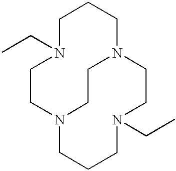

in 81% yield (1.56 g.).

This material (1.4 g.) was dissolved in anhydrous acetonitrile (50 mL). The milky white suspension was placed under vacuum until the suspension boiled and then the reaction vessel was flushed with argon. This degassing was performed 6 times. Manganese (II) chloride (0.590 gm.) was added and the reaction was refluxed for 3 hours with vigorous stirring. This was followed by immediate vacuum filtration using Whatman ® glass fiber filter paper. The dark filtrate was then evaporated under reduced pressure at 45° C. to give a brown solid. This solid was then suspended in 50 ml. toluene, and the dark brown supernatant decanted off. This washing was repeated five times. The remaining tan solid was dried under 0.05 mm vacuum overnight. This resulted in a tan solid product having the formula

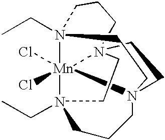

in 73% yield (1.48 gm.).

Preparation of Dichloro 4 ,11 -Dimethyl-1,4,8,11 -tetraaza-bicyclo [6.6.2]hexadecane Manganese(II)

Freshly distilled 4,11 -dimethyl- 1,4,8, 11 -tetraaza-bicyclo [6.6.2] hexadecane (25.00 g.), was dissolved in dry acetonitrile (900 mL) and the solution was degassed by applying a vacuum to the room temperature solution until it boiled and then venting with argon (repeated six times). Manganese chloride (11.25 g.) was added under argon. The cloudy reaction solution was stirred 4 hrs. under reflux becoming dark brown with suspended fine particulates. The reaction solution was filtered through a 0.2μ filter under argon. This light tan filtrate was evaporated to dryness in vacuo. The resulting tan solid was suspended in toluene (100 mL) and heated to reflux. The toluene was decanted off and the procedure repeated with another 100 mL of toluene. The balance of the toluene was removed in vacuo. After drying overnight at 0.05 mm at room temperature, a light blue solid product have the formula

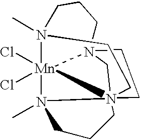

is collected, 93.5% yield (31.75 g.).

### Formulations

The MRI agents of the present invention can be in any form, for example, a solid which is dissolved in a suitable carrier prior to use, or as a pre-made solution. When in the form of a solution, a wide range of concentrations is possible depending upon the desired dosing and method of introduction into tissue.

When the MRI agents of the present invention are provided as solids, they may be in a form which will exchange one or two ligands with the carrier, typically water. For example, the MRI imaging agent 4,11-dimethyl-1,4,8,11-tetraaza-bicyclo [6.6.2] hexadecane manganese (II) diaquo may be formed in solution by adding 4,11 -dimethyl-1,4,8,11 -tetraazabicyclo[6.6.2] hexadecane manganese (II) dichloride to a saline solution prior to use.

The compositions of the present invention comprise:

a) from about 0.01% to about 99.99%, in another embodiment from 1% to about 50%, wherein another embodiment comprises from about 10% to about 75% by weight, of one or more MRI agents described herein above;

b) the balance carriers and other adjunct ingredients.

One embodiment relates to an aqueous solution of an MRI agent, said solution comprising:

a) from about 25% to about 75% by weight, of one or more MRI imaging agents described herein above;

b) the balance water.

The carriers and adjunct ingredients which comprise the balance of the pharmaceutical compositions of the present invention can be any pharmaceutically acceptable ingredient, for example, as a carrier distilled water. For embodiments wherein the imaging agent is provided as a solid which is reconstituted with water prior to use, the balance may comprise an inert filler. Or a suitable surfactant, anti-oxidant, or other stabilizer may be utilized.

### Method of Use

The present invention further relates to a method for providing enhanced human and animal tissue differentiation by contrast imaging, wherein the MRI agents of the present invention are taken up by tissue. The method of the present invention relates to establishing a blood serum level which is an effective amount of an MRI agent as described herein.

A method for providing to tissue an MRI imaging agent thereby enabling differentiation of human or animal tissue, said method comprising the step of:

A) providing to a human or animal an effective amount of an MRI agent which provides a contrast between tissues; and

B) sustaining said effective amount of MRI agent for a period of time exceeding one hour.

The serum levels for effective imaging will vary depending upon the uptake by the recipient, the type of tissue which is being targeted, and the lipophilicity of the MRI agent. In one embodiment the blood levels of the imaging agent are from about 0.001 moles to about 2 moles per liter, in another embodiment from about 0.03 moles to about 1.0 moles per liter of one or more contrasting agents according to the present invention, for example, 4,11 -dimethyl-1,4,8,11 -tetraaza-bicyclo [6.6.2] hexadecane manganese (II) diaquo complex. Another further embodiment comprises from about 0.01 to about 0.5 moles per liter of said complex. One embodiment is a pharmaceutical composition further comprising one or more carriers and adjunct ingredients.

Administration of the MRI contrast agent of the present invention to a subject, human or otherwise, on whom magnetic resonance imaging is to be performed is achieved by conventional procedures known in by those of ordinary skill in the art and disclosed in the literature. Aqueous solutions of the agent are most conveniently used. The concentration of the agent in these solutions and the amounts administered may vary widely, the optimum in each case determined by the strength of the magnetic moment of the manganese atom, the contrast enhancement strength of the chelate as a whole and the method of administration, the degree of contrast enhancement desired or needed, and the age, weight, and condition of the subject to whom administration is made. In most cases, the best results are obtained with a blood serum concentration from about 0.05 moles to about 2.0 moles, in another embodiment from about 0.1 moles to about 1.0 moles per liter blood volume. Likewise, best results in most cases are usually obtained with dosages ranging from about 0.01 mmol, preferably from about 0.05 mmol to about 1 mmol, preferably to about 0.05 mmol of agent per kilogram of whole body weight for humans (mM/kg). Administration may be achieved by any parentreral route or method, most notably by intravenous administration. The rate of administration may also vary, best results generally being obtained at rates ranging from about 0.1 mM/min/kg to about 1.0 mM/min/kg.

WHAT IS CLAIMED IS:

1. A pharmaceutical composition comprising:
a) from about 0.01% to about 99.99% by weight, of a 1,4,8,11-tetraaza-bicyclo [6.6.2] hexadecane manganese (II) complex magnetic resonance imaging agent selected from the group: 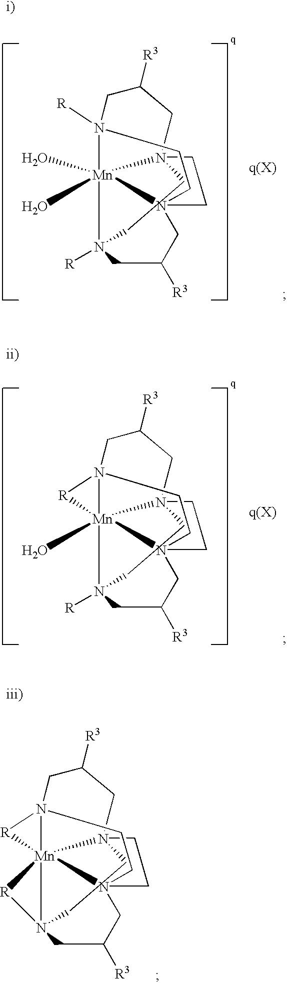
iv) and mixtures thereof;
wherein each R is independently selected from the group consisting of:
i) C₁—C₁₈ hydrocarbyl;
ii) —(CH₂)ₙCO₂—;
iii) CH₃(CH₂)ₙCO—;
iv) —(CH₂)ₙR¹;
v) —(CH₂)ₙOPO₃^{—};
vi) —[(CH₂)ₙOPO₃R²(phenyl)₂]^{—};
R¹ is hydroxyl, 2-hydroxyphenyl, 2-pyridyl, 2-furfuryl, and mixtures thereof; R² is C₁—C₁₂ linear, branched, or cyclic alkylene;
R³ is selected from the group consisting of:
i) hydrogen;
ii) C₁—C₁₈ hydrocarbyl;
iii) —OH;
iv) —(CH₂)ₘCO₂—;
v) —O(CH₂)ₘCO₂—;
vi) and mixtures thereof;
the indices m and n have the value from 0 to about 10; X is an pharmaceutically compatible anion in sufficient amount q to provide electronic neutrality; and
b) the balance carriers and other adjunct ingredients.

2. A composition according to claim 1 comprising from about 1 % to about 50%, of said imaging agent.

3. A composition according to claim 1 comprising from about 10% to about 75% by weight, of said imaging agent.

4. A composition according to claim 1 wherein said carrier is an inert solid.

5. A composition according to claim 1 wherein R is selected from the group consisting of methyl, ethyl, isopropyl, butyl, and mixtures thereof.

6. A composition according to claim 1 wherein at least one R unit comprises —(CH₂)ₙCO_{2 —}, n is from 1 to 4.

7. A composition according to claim 6 wherein n is 1.

8. A composition according to claim 1 wherein each R unit comprises —(CH₂)ₙCO_{2—}, n is from 1 to 4.

9. A composition according to claim 1 wherein said complex has the formula: 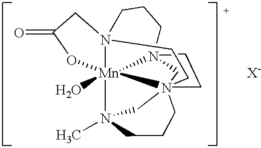
wherein X is a pharmaceutically acceptable salt.

10. A composition according to claim 1 wherein said complex has the formula:

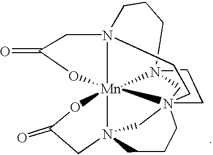

11. A pharmaceutical composition comprising an MRI agent having the formula selected from the group consisting of: 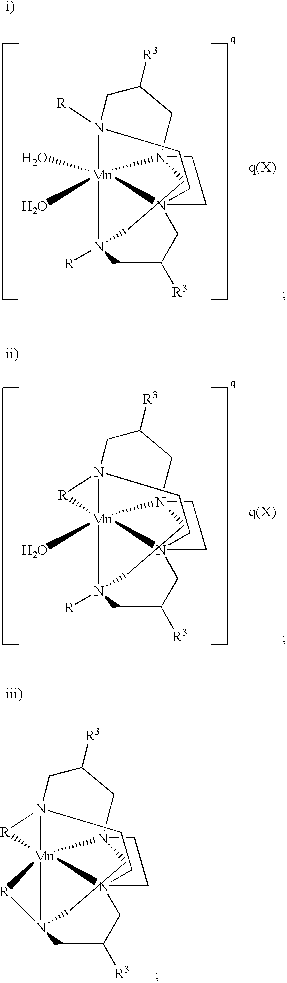
iv) and mixtures thereof;
wherein each R is independently selected from the group consisting of:
i) C₁—C₁₈ hydrocarbyl;
ii) —(CH₂)ₙCO₂—;
iii) CH₃(CH₂)ₙCO—;
iv) —(CH₂)ₙR¹;
v) —(CH₂)ₙOPO₃^{—};
vi) —[(CH₂)ₙOPO₃R²(phenyl)₂]^{—};
R¹ is hydroxyl, 2-hydroxyphenyl, 2-pyridyl, 2-furfuryl, and mixtures thereof; R² is C₁—C₁₂ linear, branched, or cyclic alkylene;
R³ is selected from the group consisting of:
i) hydrogen;
ii) C₁—C₁₈ hydrocarbyl;
iii) —OH;
iv) —(CH₂)ₘCO₂—;
v) —O(CH₂)ₘCO₂—;
vi) and mixtures thereof;
the indices m and n have the value from 0 to about 10; X is an pharmaceutically compatible anion in sufficient amount q to provide electronic neutrality.

12. A composition according to claim 11 wherein at least one R unit comprises —(CH₂)ₙCO₂—, n is from 1 to 4.

13. A composition according to claim 12 wherein n is 1.

14. A composition according to claim 11 wherein each R unit comprises —(CH₂)ₙCO_{2—}, n is from 1 to 4.

15. A method for providing an enhanced magnetic resonance image contrast in human or animal tissue, said method comprising the step of administering to a human an effective amount, of a composition comprising:
a) from about 0.01% to about 99.99% by weight, of a 1,4,8,11 -tetraaza-bicyclo[6.6.2] hexadecane manganese (II) complex magnetic resonance imaging agent selected from the group: 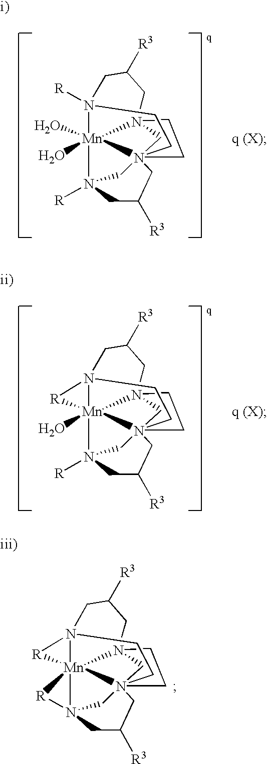
iv) and mixtures thereof;
wherein each R is independently selected from the group consisting of:
i) C₁—C₁₈ hydrocarbyl;
ii) —(CH₂)ₙCO_{2—};
iii) CH₃(CH₂)ₙCO—;
iv) —(CH₂)ₙR¹;
v) —(CH₂)ₙOPO₃^{—};
vi) —[(CH₂)ₙOPO₃R²(phenyl)₂]^{—};
R¹ is hydroxyl, 2-hydroxyphenyl, 2-pyridyl, 2-furfuryl, and mixtures thereof; R² is C₁—C₁₂ linear, branched, or cyclic alkylene;
R³is selected from the group consisting of:
i) hydrogen;
ii) C₁—C₁₈ hydrocarbyl;
iii) —OH;
iv) —(CH₂)ₘCO_{2—};
v) —O(CH₂)ₘCO_{2—};
vi) and mixtures thereof;
the indices m and n have the value from 0 to about 10; X is an pharmaceutically compatible anion in sufficient amount q to provide electronic neutrality; and
b) the balance carriers and other adjunct ingredients.

16. A method according to claim 15 wherein the serum blood levels of said agent is from about 0.001 moles to about 2 moles per liter.

17. A method for providing an enhanced magnetic resonance image contrast in human or animal tissue, said method comprising the step of:
A) administering to a human an effective amount of a composition comprising:
a) from about 0.01% to about 99.99% by weight, of a 1,4,8,11-tetraaza-bicyclo[6.6.2] hexadecane manganese (II) complex magnetic resonance imaging agent selected from the group: 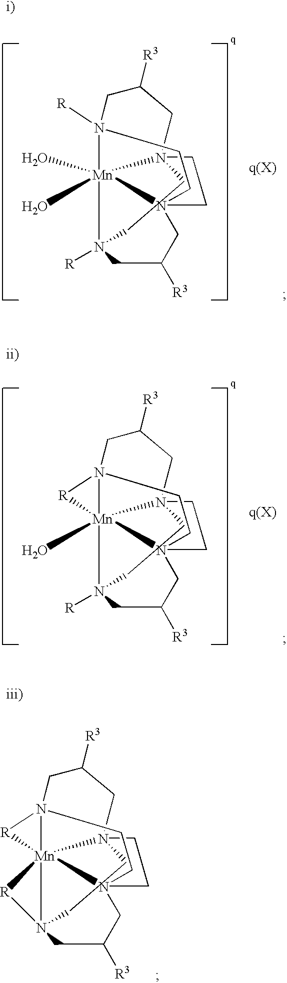
iv) and mixtures thereof;
wherein each R is independently selected from the group consisting of:
i) C₁—C₁₈ hydrocarbyl;
ii) —(CH₂)ₙCO₂—;
iii) CH₃(CH₂)ₙCO—;
iv) —(CH₂)ₙR¹;
v) —(CH₂)ₙOPO_{3—};
vi) —[(CH₂)ₙOPO₃R²(phenyl)₂]^{—};
R¹ is hydroxyl, 2-hydroxyphenyl, 2-pyridyl, 2-furfuryl, and mixtures thereof; R² is C₁—C₁₂ linear, branched, or cyclic alkylene;
R³ is selected from the group consisting of:
i) hydrogen;
ii) C₂—C₁₈ hydrocarbyl;
iii) —OH;
iv) —(CH₂)ₘCO₂—;
v) —O(CH₂)ₘCO₂—;
vi) and mixtures thereof;
the indices m and n have the value from 0 to about 10; X is an pharmaceutically compatible anion in sufficient amount q to provide electronic neutrality; and
b) the balance carriers and other adjunct ingredients; and
B) sustaining said effective amount of MRI agent for a period of time exceeding one hour.

18. A method according to claim 17 wherein at least one R unit comprises —(CH₂)ₙCO₂—, n is from 1 to 4.

19. A method according to claim 18 wherein n is 1.

20. A method according to claim 17 wherein each R unit comprises —(CH₂)ₙCO₂—, n is from 1 to 4.
